(19) United States
(12) Patent Application Publication
(10) Pub. No.: US 2009/0149923 A1
(43) Pub. Date: Jun. 11, 2009

(54) METHOD FOR EQUI-DOSED TIME FRACTIONATED PULSED UVA IRRADIATION OF COLLAGEN/RIBOFLAVIN MIXTURES FOR OCULAR STRUCTURAL AUGMENTATION

(75) Inventor: Satish V. Herekar, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: 21X Corporation dba PriaVision, Inc., Menlo Park, CA (US)

(21) Appl. No.: 12/273,444

(22) Filed: Nov. 18, 2008

Related U.S. Application Data
(60) Provisional application No. 61/012,333, filed on Dec. 7, 2007.

(51) Int. Cl. A61N 5/06

(52) U.S. Cl. 607/88

(57) ABSTRACT

Equi-dosed time-fractionated pulsed UVA is employed to irradiate a class of riboflavin/collagen mixture in the presence of copious oxygen to cause rapid crosslinking causing gelation of the riboflavin/collagen mixture in situ and to effect adhesion to underlying structure specifically ocular tissue such as scleral and corneal tissue. Irradiation according to an embodiment of the invention results in depletion of dissolved oxygen at a rate inversely related to irradiance and more particularly depletion of dissolved oxygen occurs rapidly during the process of generation/cross-linking of reactive oxygen species (ROS), specifically singlet oxygen, such that the pulsed fractionation of UVA radiation exposure increases cross-linking efficiency by allowing the re-diffusion of oxygen during pauses in exposure.

### CROSS-REFERENCES TO RELATED APPLICATIONS

The present application claims benefit under 35 USC 119(e) of U.S. provisional Application No. 61/012,333, filed on Dec. 7, 2007 entitled “Method For Equi-Dosed Time Fractionated Pulsed UVA Irradiation Of Collagen/Riboflavin Mixtures For Ocular Structural Augmentation,” the content of which is incorporated herein by reference in its entirety.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

The present invention relates generally to biomedical techniques. More particularly, the invention relates to an improved method for augmenting corneal, scleral and retinal ocular tissue and for treating, such as by repairing and reshaping, ocular tissues and eventually for refractive surgery.

Corneal and other ocular structural weakness, such as scleral structural weaknesses, can have several origins, including genetic, iatrogenic, accidents and shortcoming of desired surgical correction. Furthermore, ulcerations, melts and the like may require localized repair. Refractive corrections may comprise corneal reshaping surgery or addition of prosthetics (inlays/onlays/cavity augmentations) or some combination thereof. Localized repair is currently performed by lamellar surgery, which requires precise in situ “fitting” of biocompatible host and donor tissues and maintenance of smooth interfaces and biocompatibility thereafter, all of which are not insignificant issues. Complications from laser-based surface shaving surgery are well-known. Suturing has its own set of difficulties and shortcomings, as does tissue gluing.

It is long known that collagen exposed to riboflavin, also known as vitamin B2, in the presence of ultraviolet light produces cross-linking, which is useful as a cell scaffold for rebuilding cartilaginous defects. It is also known that corneal tissue can be stiffened by cross-linking by UVA irradiation in the presence of riboflavin eye drops. However, problems exist with known techniques of riboflavin-mediated cross-linking in collagen, including undesired shrinkage and increased opacity. (Representative citations herein are not necessarily prior art.) In the ocular domain, as published since the filing of the priority application upon which this invention is based, work has been reported on techniques for corneal cross-linking by photopolymerization of stromal fibers in the presence of riboflavin by irradiation with ultraviolet light. See Cosimo Mazzotta PhD, Angelo Balestrazzi PhD, Stefano Baiocchi PhD, Claudio Traversi MD PhD, Aldo Caporossi MD (2007), “Stromal haze after combined riboflavin-UVA corneal collagen cross-linking in keratoconus: in vivo confocal microscopic evaluation,” Clinical & Experimental Ophthalmology, Volume 35 (6), pp. 580-582, (August 2007). (doi:10.1111/j.1442-9071.2007.01536.x). No separate application of augmentation materials or suggestions of mixtures of photoreactive augmentation materials was reported in that study. Moreover, the researcher reported negative results: The therapy caused stromal haze after the cross-linking treatment.

Others have reported on the results of collagen cross-linking induced by riboflavin exposed to UVA. Wollensak reported on collagen cross-linking induced by riboflavin UVA and involving injection of a polymeric composition forming a gel into the eye in The Journal of Cataract & Refractive Surgery, Vol. 30 (3), pp. 689-695 (March 2004). Augmentation by onlay in particular was not addressed.

In work by the present inventor (not as prior art) identified in Provisional Patent Application No. 60/869,048 filed Dec. 7, 2006, and now found in Non-Provisional patent application Ser. No. 11/952,801 filed Dec. 7, 2007, a method and material for in situ corneal structural augmentation was disclosed involving continuous irradiation of collagen/riboflavin mixtures for periods of several minutes.

There remains in the art a need for a method for effective and more rapid augmentation therapy for ocular applications.

### SUMMARY OF THE INVENTION

According to the invention, equi-dosed time-fractionated pulsed UVA is employed to irradiate a class of riboflavin/collagen mixture in the presence of copious oxygen to cause rapid cross-linking resulting in gelation of the riboflavin/collagen mixture in situ and to effect adhesion to underlying structure, specifically ocular tissue such as scleral and corneal tissue. Irradiation according to an embodiment of the invention results in depletion of dissolved oxygen at a rate inversely related to irradiance and more particularly depletion of dissolved oxygen occurs rapidly during the process of generation/cross-linking of reactive oxygen species (ROS), specifically singlet oxygen, such that the pulsed fractionation of UVA radiation exposure increases cross-linking efficiency by allowing the re-diffusion of oxygen during pauses in exposure. An intended application is structural augmentation of ocular tissue, as may be used for better stabilizing progressive corneal diseases, such as keratoconus (KCN), ectasia, ulcers/melts and the like.

The invention has identified the required presence of dissolved corneal oxygen (pO₂) in the cross-linking/ROS generation process and suggests that UVA fractionation and optionally, UVA pulsing, may lead to significantly stronger (at depth) and safer collagen cross-linking in a shorter time.

The invention will be better understood by reference to the following drawing and related description.

### BRIEF DESCRIPTION OF THE DRAWINGS

FIG. 1 is a block diagram of an experimental device for testing efficacy of corneal augmentation according to the invention.

FIG. 2 is a graph illustrating relative stiffness of cornea under various treatments, including treatment according to the invention.

### DESCRIPTION OF SPECIFIC EMBODIMENTS OF THE INVENTION

According to a specific embodiment of the invention, a collagen/riboflavin mixture is irradiated in a specific pattern with UVA with a specific pattern of pulses at a fractionated duty cycle in the presence of oxygen to generate reactive oxygen species and to cause desired forms of gelation, that is, gelation with robustness in terms of stability, longevity, rigidity, optical clarity, low shrinkage and high adhesion to a substrate of ocular tissue. Decreasing the time of UVA exposure or minimizing the UVA intensity in a therapy according to the invention tends to minimize undesired cellular changes during augmentation or generation of in situ collagen gels.

Modulating exposure affects the nature of the gels formed. Using equi-dosed conditions as a modus, UVA is applied in time-fractionated pulses to collagen/riboflavin mixtures in the form of amorphous gels. Collagen/riboflavin mixtures that were prepared as previously described in Provisional Patent Application 60/869,048 filed Dec. 7, 2006, and its corresponding Non-Provisional Patent Application entitled “Method And Material For In Situ Corneal Structural Augmentation” in the name of the present inventor (U.S. patent application Ser. No. 11/952,801) using a 6% bovine collagen solution at a pH of 5.5 and 6.5 containing riboflavin-based cross-linker in a ratio of 5:100, although a wide range of concentration mixtures are contemplated and have a significant effect on rapidity of gelation.

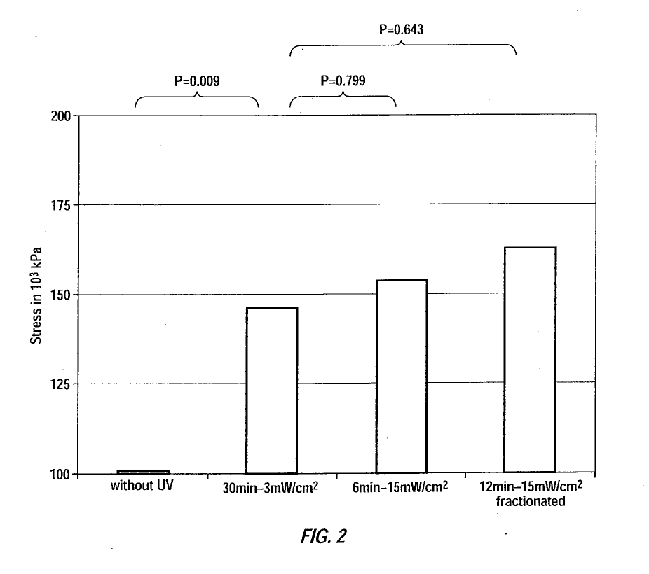

According to an embodiment of the present invention, the collagen/riboflavin mixture is rapidly gelated to an intended robustness by exposing the mixture in the presence of oxygen to a fractionated dosage of pulsed UVA, thereby generating reactive oxygen species of singlet oxygen that has beneficial outcomes. The UVA is at an instantaneous fluence (intensity per sq. cm.) of between 1 mW/cm² and 30 mW/cm² and preferably at a nominal optimal value of 15 mW/cm² during the ON portion of the duty cycle, which may vary from 1% to 100% for experimental purposes but less than 100% for actual operation and preferably for a period of a few seconds on at a nominal optimal duty cycle of 20% or 1:5 with OFF time of approximately 30 seconds over a period of 6 minutes. However, a duty cycle of between 2:1 (50% on) and 3:1 (67% on) with off time of about 30 seconds over a period of 12 minutes has been employed effectively in experiment. The presence of adequate oxygenation impacts efficacy as herein explained. For experimental purposes, this treatment was compared to continuous UVA radiation exposure at a much lower intensity of 3 mW/cm² for a much longer period of 30 minutes. The results were notably better in procedures according to the invention, as herein reported. (See Table 1 and FIG. 2.)

The properties of the resulting gels were compared to determine efficacy, including, among other things, denaturation, longevity and adhesion. Denaturation temperatures were used as an indication of the degree of cross-linking. These were found to be similar among the gels prepared. It has been determined that higher intensity irradiation for shorter periods of exposure time results in increased cross-linking.

Measurements of reactive oxygen species (ROS), namely singlet oxygen (¹O₂), in the course of irradiation, correlate with the discovery that continued exposure to low dose UVA leads to lower levels of reactive oxygen species over a shorter time period than higher intensities for shorter time periods. Higher levels of reactive oxygen species have desired effects.

Mechanical tests demonstrate that there is an advantage to using the higher intensity/shorter duration cross-linking methodology. Mechanical properties of gels formed in this manner were almost 25% higher (e.g., more rigid gelation) than those prepared using the lower dose/longer duration (non-pulsed) protocol.

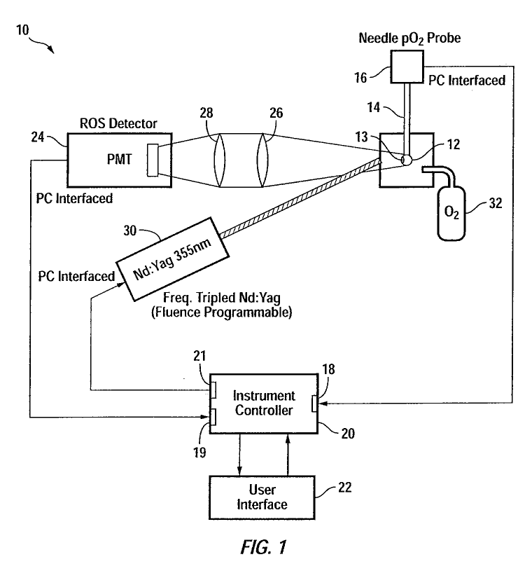

FIG. 1 is a block diagram of an experimental apparatus 10 according to an embodiment of the invention. It can be generalized to a therapeutic device with appropriate modifications. A target 12, as for example an eye in situ, defines a volume of interest. For experimental purposes a needle probe 14 engages the target to measure oxygen levels at depth, and it is coupled via a transducer 16 to an input 18 of an instrument controller 20, which is under the control of a user interface 22. A photomultiplier tube (PMT) detector 24 is coupled to observe the target 12 through focusing lenses 26, 28. The PMT detector 24 is for example a fast IR sensitive device operative as a reactive oxygen species (ROS) detector, since ROS levels are an indication of progress of treatments according to the invention. The PMT detector 24 is coupled to the instrument controller 20 through an input interface 19. The instrument controller 20 is coupled via a driver interface 21 to a controllable IR radiation source, such as an IR laser, specifically a fluence programmable frequency tripled ND:Yag laser 30 operative at 355 nm output and preferably directed at the collagen/riboflavin mixture 13 at the target volume 12. It is not necessary that the radiation be coherent in practice, so long as the fluence is homogenous (such as collimated and/or focused), permitting the control of irradiance of a target region. The target volume 12 may comprise the mixture, or the mixture may be a surface volume in situ on ocular tissue, or both. The instrument controller 20 is configured to generate control signals causing the laser 30 or other appropriate UVA source to deliver pulsed IR irradiance with fluence at an equi-dosed level at the selected pulse rate for a fractionated period of a selected duty cycle for a selected duration. The irradiance is delivered in the presence of free oxygen of at least ambient levels. The amount of dissolved oxygen in the target volume is not sufficient for these purposes, so under the present methodology, both free oxygen is provided and time is allowed to permit rediffusion of oxygen into the target volume. The free oxygen may be supplied from a controlled oxygen source 32 or if it is determined that ambient air is sufficiently oxygen rich and uncontaminated, it may react from the ambient atmosphere. The PMT detector 24 is used for experimental purposes and is operative to detect reactive oxygen species in response to irradiance of the mixture 13 for feedback and/or output recording and display via the instrument controller 20. A PMT detector 24 is not needed where there is known sufficient oxygen and the instrument is calibrated, etc. UVA levels may be monitored at the source.

With colleagues in several countries, and using the apparatus 10, the inventor has initiated testing of nomograms that included such variables as UVA irradiance, oxygenation, and exposure time with the aim of measuring differences in collagen thermo-mechanical strength/cross-link densities by differential scanning calorimetry (DSC), stromal cell viability, depth of effect-confocal, stress/strain extensiometry and optical transmission/clarity while maintaining equal UVA dosages. The tests were performed on “cornea-mimicking” cross-linkable collagen/Riboflavin (0.1%) liquid samples (“PriaGels”—a trademark for reagents from the assignee of the present invention). Bovine collagen was diluted to a concentration of 30 mg/ml and cross-linked by UVA exposure at 370 nm in a UV oven (Cure Zone Control-2-Cure) in the presence of 0.1% Riboflavin under various conditions. Specifically, the high irradiance nomogram was set at 15 mW/cm² for a period of 6 minutes while the low irradiance setting was a the current standard of 3 mW/cm² for a period of 30 minutes, both dosing to a value of 5.4 J/cm² and with a two times safety margin for keratocyte and endothelial cell apoptosis thresholds (i.e., actual exposures are half of apoptosis thresholds). The fractionation periods were set for 1 minute ON and OFF. (Duty cycle 2:1) Gel integrity was assessed by examination of mechanical properties. Measurement of collagen denaturation temperatures by differential scanning calorimetry (DSC) was also performed as an indirect measurement of the relative collagen cross-linking density of the gels. Gel shrinkage measurement was also used for comparison. To examine the effects of these dose conditions, human corneal stromal cells cultured on tissue culture polystyrene were exposed to identical UVA conditions. The thermal stability of these gels was assessed by exposure to elevated temperatures (80° C.) for periods of 30 seconds.

Experiments were undertaken to estimate singlet oxygen baseline lifetimes and rate of dissolved oxygen consumption under two irradiances using commercial Riboflavin, as well as proprietary Riboflavin formulations. The setup included, briefly: a frequency tripled YAG (355 nm) laser for time-resolved UVA excitation, a micro-needle dissolved oxygen sensor (pO₂), and an infrared (1270 nm) sensitive, fast (˜ns) PMT photon counter/sensor monitoring luminescence in a small volume in a cuvette sample containing collagen and 0.1% Riboflavin. (Other configurations were not excluded but were not fully explored during the proof of concept stage of testing.)

Results of these experiments are summarized in Tables 1 and 2 below.

TABLE 1
Summary of equi-dosed gels with high/low UVA irradiance WITHOUT
fractionation

| Irradiance | Time | Denaturation | Shrinkage | Modulus | Max | Stress/Strain |
|---|---|---|---|---|---|---|
| (mW/cm²) | (min) | T* (° C.) | T (° C.) | (MPa) | Stress (MPa) | Pig eyes* |
| 15 | 6 | 56.4 | 53.0 | 0.43 +/− 0.21 | 0.09 +/− 0.01 | 180 |
| 3 (prior art) | 30 | 52.1 | 47.0 | 0.17 +/− 0.04 | 0.07 +/− 0.01 | 150 |

Note:
*Minimal levels of cell death were visually observed under both levels of UVA exposure. Exposure of the gels to high temperature for a period of 4 seconds altered the low irradiance/long exposure time gels slightly but did not alter the transparency or mechanical properties of the high irradiance/short exposure time gels. Differences in mechanical properties and in denaturation temperatures were statistically significant (p < 0.05).

TABLE 2
Summary of ROS (¹O₂) lifetimes, dissolved oxygen pO₂ vs. time:

| | pO₂ Time | | | | |
|---|---|---|---|---|---|
| | (min)* | | | | |
| Irradiance | to 50% | PriaGel* | PriaVision** | TUD** | IROC** |
| (mW/cm²) | crossover | t (μsec) f:pO₂ | t (μsec) | t (μsec) | t (μsec) |
| 15 | 0.8 | 10-20 | 4.5 | 3.8 | 4.3 |
| 3 | 4.0 | Same | Same | Same | Same |

Notes:
*Aerated 0.1% Riboflavin and collagen in a closed cuvette (CC)
**Reference to sources of Riboflavin suppliers. TUD = Technical University of Dresden, IROC = Institute for Refractive and Opthalmo Surgery of Switzerland. Common content: aerated 0.1% Riboflavin (CC). The collagen source was Inamed/Allergan.

UVA fractionated cross-linking experiments showed an increase in denaturation temperature of approximately 5° C. relative to equal-irradiance/equal-dosed exposures with the one minute “dark cure” fractionation period. High irradiance but non-fractionated increases were 4° C. over low irradiance non-fractionated exposed gels. In comparison, chemical cross-linking with EDC/NHS resulted in a denaturation temperature of approximately 55° C., suggesting maximal cross-linking with the inventive technique as well. The denaturation temperatures were found to be stable for at least 45 days post cross-linking.

In summary, the results demonstrate that a high irradiance (˜15 mW/cm2) nomogram is preferable for improved thermo-mechanics, and shortened treatment time, given conserved corneal transparency and stromal keratocyte survivability and furthermore, UVA fractionation enhanced this thermo-mechanical/XL density effect further.

Summarizing the results of ROS singlet oxygen (¹O₂) lifetimes, dissolved oxygen consumption (pO₂), and UVA vs. irradiance linearity (Table 2), and UVA vs. 0.1% Riboflavin, ROS ¹O₂ generation linearity was very high (R2=0.999) and rapid (<2 μs). From Table 2, it is seen that IROC 0.1% Riboflavin ROS (¹O₂) concentration was reduced by about 25% compared to TUD and PriaVision materials. However, this is likely due to nitrogen packing of the IROC vials. This may not be clinically relevant due to the high Riboflavin quantum efficiencies.

The following conclusions have been drawn from these experiments: ROS (¹O₂) generation is rapid, and slow re-diffusion of local dissolved oxygen needs continuous replenishment in exposed tissue volumes. Experiments show that ROS (¹O₂) generation takes about 1 μs upon UVA excitation, and ROS (¹O₂) lifetimes are of the order of 10-20 μs, depending on the dissolved oxygen concentration. These radicals activate amine functional groups present in collagen to subsequently form intra-molecular bonds in a reaction that may take about 10-20 μs and is very local with almost no diffusion. As the stroma becomes increasingly hypoxic, UVA with Riboflavin exposure generates increasing amounts of Type I radicals and lesser amounts of Type II singlet oxygen (¹O₂) radicals that are the predominant reactive drivers for collagen cross-linking, thus increasing keratocyte apoptosis through cell peroxidation. Repopulation of possibly diseased stromal cells following apoptosis may play an as-yet unknown but beneficial role in the overall outcomes more than 6 months post operatively.

Ambient oxygen re-diffusion is slow. Consistent with results reported by others, a normal epithelialized cornea requires typically 45 seconds to attain steady state posterior corneal concentration for an ambient step in oxygen concentration. Stromal de-oxygenation times (from >90% to <10% of ambient) are likely to be on the order of 10 seconds with the inventive high UVA irradiance nomogram or 50 seconds with the prior-art low UVA irradiance for targeted corneal volumes of 40 mm³.

It is likely that insufficient ROS (¹O₂) generation is the cause of stronger anterior cross-linking than posterior cross-linking, especially given the full thickness keratocyte apoptosis, indicating a hydrogen peroxide Type I radical generation in the posterior region. High Riboflavin quantum efficiencies with oxygen also lends to this effect, as rapid oxygen depletion without re-diffusion is the anticipated state during the standard (inefficient) exposure. Three schemes were considered for mitigating this hypoxic state: 1) a hyperbaric corneal environment, 2) UVA fractionation to allow re-diffusion from ambient air supply or from an ancillary oxygen supply, and 3) Exogenous drug dosing to compensate for deep stromal hypoxia. Exogenous drug dosing may be too complicated, and hyperbaric environments reportedly induce lenticular complications. Therefore, the preferred mitigating scheme, and the scheme upon which the invention is based is UVA fractionation under ambient conditions, including the presence of free oxygen.

UVA fractionated cross-linked collagen is made stronger with maximally dense cross-link formation based on thermo-mechanical properties. The depth of dense cross-links appears to be more uniform in the upper 350 μm of the material.

Stress/strain extensiometry pig eye data, when involving equi-dosage under varying irradiances, disclosed an interesting trend: Higher irradiance stress/strain moduli are statistically similar to those of 3 mW/cm²×30 minutes controls, but there is a drift towards lower modulus values presumably due to greater surface cross-linking effects and faster oxygen depletion (permitting fewer cross-links at depth). A 0.5% Riboflavin photo-sensitizer test showed a statistically significant lower modulus for similar irradiance/dosage due to highly superficial cross-linking effects (which is likely due to rapid anoxia just underneath the surface). This data is illustrated in FIG. 2.

FIG. 2 illustrates comparative stiffness based on different therapies as measured in a porcine cornea where the stress as measured is at 10% strain. Column 1 is the resultant stiffness where Riboflavin alone is used as therapy, and thus it is designated the control sample. Column 2 is the result of treatment by riboflavin plus continuous UVA at 3 mW/cm² for 30 minutes. This is the aforementioned technique known in the prior art. It results in a 45% improvement over the control. Column 3 illustrates the result of treatment by Riboflavin plus pulsed UVA at 15 mW/cm² for 6 minutes in the presence of reactive oxygen, in accordance with the techniques taught by the present invention. It shows a 55% improvement over the control. Column 4 illustrates the result of treatment by riboflavin plus pulsed UVA at 15 mW/cm² for 12 minutes in the presence of reactive oxygen, with one-minute pauses further in accordance with techniques taught by the present invention. It shows a 65% improvement over the control. There is evident improvement by each of the techniques employing UVA over that of Riboflavin therapy alone. However, due to the relatively small test sampling population, there is no claim to a significant statistical difference in the stiffness results between the various UVA therapies. Nevertheless, the technique according to the invention using high irradiance that is pulsed or pulsed and time fractionated, completes the therapy in as much as 80% less the time than the prior art UVA therapy. Moreover, pulsed, equi-dosed, time fractionated UVA therapy may well provide the most favorable results.

During cross-linking, dissolved oxygen had a significant impact on thermo-mechanical characteristics by high irradiance therapy with UVA and Riboflavin alone and more so by high irradiance therapy with fractionated UVA and Riboflavin as compared to the low irradiance non-fractionated longer exposure therapy. While low irradiance was suboptimal and appeared to maximally crosslink gels, increased dosage runs the risk of endothelial damage. In high irradiance fractionation, similar apoptosis and optical transmission characteristics were observed in all equi-dosed cases. Lack of reduction in apoptosis with high irradiance is believed to be due in part to oxygen abundance as compared to the oxygen-diffusion-constrained native stromal environment. Hypoxia-induced apoptosis under low irradiance and lengthy UVA and Riboflavin exposure may be a leading cause of some of the side effects, specifically haze, associated with this otherwise highly promising therapy.

A way to mitigate this side effect appears to be to UVA fractionate under stronger irradiance in accordance with the invention, initiating mainly Type II singlet oxygen generation deep in the stroma. UVA fractionated cross-linking with a setting of 30 secs ON and 30 secs OFF for a total 12 minutes of treatment time at 15 mW/cm² has been found to be acceptable if not optimal, based on available experimental data. A range of greater than six minutes and less than thirty minutes at this 1:1 (50%) duty cycle for pulsed irradiance is nevertheless indicated as efficacious. In case a desired goal is minimizing apoptosis, a pulsed UVA delivery during fractionation at about a ˜100+ KHz rate with less than about 1:3 duty cycle should reduce keratocyte depletion considerably.

With a goal of speeding up ROS (¹O₂) generation in an oxygenated stroma with high irradiance, UVA dosing to maintain endothelial safety, UVA fractionating to replenish stromal oxygen and UVA pulsing to minimize untimely UVA exposure, highly efficacious speed-up of corneal cross-linking with Riboflavin (CXL) should be feasible.

The preliminary studies have neither proven the benefits nor demonstrated harmful side effects if any, from reduced apoptosis, improved thermo-mechanics, and aeration-punctuated shortened exposure, but observations indicate that the potentially beneficial effects are worthy of disclosing the therapy and of seeking to establish proprietary rights.

Therefore the invention has been described with respect to specific embodiments. Other embodiments will be evident to those of skill in the art. It is therefore not intended that the invention be limited, except in accordance with the claims.

What is claimed is:

1. A method for effecting gelation of a collagen/riboflavin mixture having a controlled mixture ratio and concentration in situ on ocular tissue for enhancement of said ocular tissue, the method comprising:
irradiating the collagen/cross-linker mixture in the presence of oxygen with ultraviolet energy in equi-dosed time-fractionated pulses over an exposure period sufficient to promote the generation of reactive oxygen species and to yield gelation with desired physical robustness and adhesion to said ocular tissue.

2. The method according to claim 1 wherein the ultraviolet energy is UVA.

3. The method according to claim 2 wherein the UVA is of 355 nm frequency.

4. The method according to claim 2 wherein instantaneous fluence of said UVA is between 1 mW/cm² and 30 mW/cm².

5. The method according to claim 4 wherein treatment fractionation has an on/off duty cycle of between 1:100 and 100:1.

6. The method according to claim 4 wherein said UVA instantaneous fluence is less than 15 mW/cm² at 355 nm frequency and the pulses have pulse rate at a frequency of greater than 100 kHz with greater than a 1:3 duty cycle for a treatment fractionation duty cycle of greater than 1:5 for a treatment duration of less than 6 minutes.

7. The method according to claim 6 wherein the irradiance exposure off time is less than 30 seconds.

8. The method according to claim 4 wherein treatment fractionation has an on/off duty cycle of between 2:1 and 3:1.

9. The method according to claim 8 wherein the irradiance exposure off time is less than 30 seconds.

10. The method according to claim 4 wherein treatment fractionation has an on/off duty cycle of approximately 1:5.

11. A method for effecting gelation of a collagen/riboflavin mixture comprising:
irradiating the collagen/cross-linker mixture in a controlled ratio of concentration with ultraviolet energy in equi-dosed time fractionated pulses over an exposure period sufficient to yield gelation with desired physical characteristics.

12. An apparatus for effecting gelation of a collagen/riboflavin mixture in situ with ocular tissue comprising:
an ultraviolet laser for irradiating the collagen/cross-linker mixture in situ with ultraviolet energy; and
a controller coupled to the laser for controlling irradiance in equi-dosed time fractionated pulses at a selected fractionated duty cycle over a selected exposure period sufficient to yield gelation with desired physical characteristics.
